SEQUENCE LISTING (2 sequences)

SEQ ID NO: 1 - DNA, 1728 bp, Homo sapiens
agagaaaattgctgttgggatgaagctttgcagccttgcagtccttgtacccattgttctcttctgtgagcagcatgtcttcgcgtttcagagtggccaagttctagctgctcttcctagaacctctaggcaagttcaagttctacagaatcttactacaacatatgagattgttctctggcagccggtaacagctgaccttattgtgaagaaaaaacaagtccatttttttgtaaatgcatctgatgtcgacaatgtgaaagcccatttaaatgtgagcggaattccatgcagtgtcttgctggcagacgtggaagatcttattcaacagcagatttccaacgacacagtcagcccccgagcctccgcatcgtactatgaacagtatcactcactaaatgaaatctattcttggatagaatttataactgagaggcatcctgatatgcttacaaaaatccacattggatcctcatttgagaagtacccactctatgttttaaaggtttctggaaaagaacaaacagccaaaaatgccatatggattgactgtggaatccatgccagagaatggatctctcctgctttctgcttgtggttcataggccatataactcaattctatgggataatagggcaatataccaatctcctgaggcttgtggatttctatgttatgccggtggttaatgtggacggttatgactactcatggaaaaagaatcgaatgtggagaaagaaccgttctttctatgcgaacaatcattgcatcggaacagacctgaataggaactttgcttccaaacactggtgtgaggaaggtgcatccagttcctcatgctcggaaacctactgtggactttatcctgagtcagaaccagaagtgaaggcagtggctagtttcttgagaagaaatatcaaccagattaaagcatacatcagcatgcattcatactcccagcatatagtgtttccatattcctatacacgaagtaaaagcaaagaccatgaggaactgtctctagtagccagtgaagcagttcgtgctattgagaaaactagtaaaaataccaggtatacacatggccatggctcagaaaccttatacctagctcctggaggtggggacgattggatctatgatttgggcatcaaatattcgtttacaattgaacttcgagatacgggcacatacggattcttgctgccggagcgttacatcaaacccacctgtagagaagcttttgccgctgtctctaaaatagcttggcatgtcattaggaatgtttaatgcccctgattttatcattctgcttccgtattttaatttactgattccagcaagaccaaatcattgtatcagattatttttaagttttatccgtagttttgataaaagattttcctattccttggttctgtcagagaacctaataagtgctactttgccattaaggcagactagggttcatgtctttttaccctttaaaaaaaaattgtaaaagtctagttacctactttttctttgattttcgacgtttgactagccatctcaagcaactttcgacgtttgactagccatctcaagcaagtttaatcaaagatcatctcacgctgatcattggatcctactcaacaaaaggaagggtggtcagaagtacattaaagatttctgctccaaattttcaataaatttcttcttctcctttaaaaaaaaaaaaaaaaaaa

SEQ ID NO: 2 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQTAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

(19) United States
(12) Patent Application Publication
(10) Pub. No.: US 2005/0009024 A1
(43) Pub. Date: Jan. 13, 2005

(54) Method for the detection of a polymorphism in cpb2

(76) Inventors: John Edward Norris Morten, Macclesfield (GB); Karen Nicola Jackson, Macclesfield (GB)

Correspondence Address: FISH & RICHARDSON P.C., 225 FRANKLIN STREET, BOSTON, MA, 02110, US

(21) Appl. No.: 10/486,034

(22) PCT Filed: Aug. 6, 2002
(86) PCT No.: PCT/GB02/03626

(30) Foreign Application Priority Data
Aug. 8, 2001 (GB) 0119269.9

(51) Int. Cl. C12Q 1/68

(52) U.S. Cl. 435/6

(57) ABSTRACT

This invention relates to polymorphisms in the human CPB2 gene and corresponding novel allelic polypeptides encoded thereby. The invention also relates to methods and materials for analysing allelic variation in the CPB2 gene, and to the use of CPB2 polymorphism in treatment of diseases with CPB2 drugs.

This invention relates to polymorphisms in the human CPB2 gene and corresponding novel allelic polypeptides encoded thereby. The invention also relates to methods and materials for analysing allelic variation in the CPB2 gene, and to the use of CPB2 polymorphism in treatment of diseases with CPB2 transportable drugs.

Human plasma carboxypeptidase B2 (CPB2) is also known as carboxypeptidase U (CPU) and thrombin activable fibrinolysis inhibitor; the term CPB2 is used herein. It is an unstable basic carboxypeptidase which circulates in human plasma in its proenzyme form, procarboxypeptidase B2 (proCPB2). Vanhoof et al (1996) noted that proCPB2 exhibits affinity for plasminogen, and is converted to its active form through the action of thrombin and plasmin. Bazjar et al (1996) noted that the most likely physiological activator of proCPB2 is the thrombin-thrombomodulin complex. The deduced amino acid sequence of the protein reveals a primary translation product very similar to tissue-type carboxypeptidases A and B. Eaton et al (1991) cloned the cDNA for proCPB2. The predicted 423 amino acid protein, consists of a 22-amino acid signal peptide, a 92-amino acid activation peptide, and a 309-amino acid catalytic domain.

When activated by trypsin, it hydrolyzes carboxypeptidase B substrates, hippuryl-Arg and hippuryl-Lys, but not carboxypeptidase A substrates. It is inhibited by the specific carboxypeptidase B inhibitor (DL-5-guanidinoethyl) mercaptosuccinic acid. Tsai and Drayna (1992) demonstrated that the gene is located on human chromosome 13. The gene was regionalized by Vanhoof et al (1996) using fluorescence in situ hybridisation to 13q14.11. No genetic disorders that are likely to involve CPB2 have been mapped to the region.

A possible role for CPB2 is in the activation of plasminogen, an enzyme which catalyzes the deposition and removal of fibrin. A balance between the activities of the coagulation and fibrinolysis cascades is essential to protect from excessive blood loss upon injury and to maintain blood fluidity within the vascular system. Imbalances are characterised by either bleeding or thrombotic tendencies, the latter of which are manifested as heart attacks and strokes. Inhibition of CPB2 to accelerate fibrinolysis could be a treatment for thromboembolic disorders.

One approach is to use knowledge of polymorphisms to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

One polymorphism in the coding sequence of CPB2 has been described in data from the SNP Consortium, TSC0985620. Zhao et al, Thromb Haemost 80, 949-55, 1998 disclosed an A147T protein polymorphism under the name TAPI (equivalent to A169T herein) and a silent base change polymorphism at position 697. The present invention relates to additional polymorphisms in CPB2 and their haplotypes.

All positions herein of polymorphisms in the CPB2 polynucleotide relate to the position in SEQ ID NO 1 unless stated otherwise or apparent from the context.

All positions herein of polymorphisms in the CPB2 polypeptide relate to the position in SEQ ID NO 2 unless stated otherwise or apparent from the context.

According to one aspect of the present invention there is provided a method for the detection of a polymorphism in CPB2 in a human, which method comprises determining the sequence of the human at at least one of the following polymorphic positions:
positions 310, 549, 682 and 772 in CPB2 polynucleotide as defined by the position in SEQ ID NO: 1; and
position 177 in CPB2 polypeptide defined by position in SEQ ID NO: 2.

D at 177 is conserved between man and mouse and is part of a 26 amino acid sequence conserved between man and mouse suggesting that this is a functionally important site. Substitution of G for D substitutes a small, uncharged amino acid in place of a larger negatively charged amino acid, which is expected to affect properties of the protein.

According to one aspect of the present invention there is provided a method for the detection of a polymorphism in CPB2 in a human, which method comprises determining the sequence of the human at at least one of the following polymorphic positions:
positions 310, 524, 549, 682, 697 and 772 in CPB2 polynucleotide as defined by the position in SEQ ID NO: 1; and
positions 169 and 177 in CPB2 polypeptide defined by position in SEQ ID NO: 2.

Preferably the method comprises detection of any one of the following six haplotypes:

| | 310 | 524 | 682 | 697 | 772 | 1059 |
|---|---|---|---|---|---|---|
| Haplotype | allele | allele | allele | allele | allele | allele |
| 1 | C | G | A | C | C | T |
| 2 | C | A | G | C | C | T |
| 3 | C | G | A | C | T | C |
| 4 | T | G | A | T | C | T |
| 5 | C | G | A | C | C | C |
| 6 | C | A | G | C | C | C |

In another embodiment, the method preferably comprises detection of any one of the following four combinations of allelic variant determined amino acids based on positions identified in SEQ ID NO: 2:

| | |
|---|---|
| 1 | thr169/thr347 |
| 2 | ala169/thr347 |
| 3 | thr169/ile347 |
| 4 | ala169/ile347 |

The term human includes both a human having or suspected of having a CPB2 mediated disease and an asymptomatic human who may be tested for predisposition or susceptibility to such disease. At each position the human may be homozygous for an allele or the human may be a heterozygote.

The term polymorphism includes single nucleotide substitution, nucleotide insertion and nucleotide deletion which in tie case of insertion and deletion includes insertion or deletion of one or more nucleotides at a position of a gene and corresponding changes in any polypeptide encoded thereby.

The method for nucleotide sequence detection is preferably one in which the sequence is determined by a method selected from amplification refractory mutation system and restriction fragment length polymorphism.

The status of the individual may be determined by reference to allelic variation at any one, two, three, four, five, six, seven, eight or more positions.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et. al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| CPB2 | Human plasma carboxypeptidase B2 |
| DGGE | Denaturing gradient gel electrophoresis |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

TABLE 1
Mutation Detection Techniques

General: DNA sequencing, Sequencing by hybridisation
Scanning: PTT*, SSCP, DGGE, TGGE, Cleavase, Heteroduplex
analysis, CMC, Enzymatic mismatch cleavage
*Note:
not useful for detection of promoter polymorphisms.

Hybridisation Based

Solid phase hybridisation: Dot blots, MASDA, Reverse dot blots, Oligonucleotide arrays (DNA Chips)

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B1 (Zeneca Limited), COPS—Gibbs et al (1989), Nucleic Acids Research, 17, 2347.

Incorporation Based: Mini-sequencing, APEX

Restriction Enzyme Based: RFLP, Restriction site generating PCR

Ligation Based: OLA

Other: Invader assay

TABLE 2
Signal Generation or Detection Systems

Fluorescence: FRET, Fluorescence quenching, Fluorescence
polarisation - United Kingdom Patent No. 2228998 (Zeneca Limited)
Other: Chemiluminescence, Electrochemiluminescence, Raman,
Radioactivity, Colorimetric, Hybridisation protection assay,
Mass spectrometry

TABLE 3
Further Amplification Methods

SSR, NASBA, LCR, SDA, b-DNA

TABLE 4
Protein variation detection methods

Immunoassay
Immunohistology
Peptide sequencing

Preferred mutation detection techniques include ARMS™, ALEX™, COPS, Taqman, Molecular Beacons, RFLP, and restriction site based PCR and FRET techniques. Immunoassay techniques are known in the art e.g. A Practical Guide to BLISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom. Histological techniques are described in Theory and Practice of Histological Techniques by J D Bancroft & A Stevens, 4^{th} Edition, Churchill Livingstone,1996. Protein sequencing is described in Laboratory Techniques in Biochemistry and Molecular Biology, Volume 9, Sequencing of Proteins and Peptides, G Allen, 2^{nd} revised edition, Elsevier, 1989.

Particularly preferred methods include ARMS™ and RFLP based methods. ARMS™ is an especially preferred method.

In a further aspect, there is provided use of a method as defined herein to assess the pharmacogenetics of a CPB2 drug.

Assays, for example reporter-based assays, may be devised to detect whether one or more of the above polymorphisms affect transcription levels and/or message stability and/or drug binding. Potential drugs may be screened to test if polymorphic variation in CPB2 has any affect on drug performance such as, for example, dosage or efficacy.

Individuals who carry particular allelic variants of the CPB2 gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The methods of the invention may be useful both to predict the clinical response to such agents and to determine therapeutic dose.

In a further aspect, the methods of the invention, are used to assess the predisposition and/or susceptibility of an individual to diseases mediated by CPB2. This may be particularly relevant in the development cardiovascular disease and the present invention may be used to recognise individuals who are particularly at risk from developing these conditions.

In a further aspect, the methods of the invention are used in the development of new drug therapies which selectively target one or more allelic variants of CPB2. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further aspect of the invention the presence or absence of variant nucleotides is detected by reference to the loss or gain of, optionally engineered, sites recognised by restriction enzymes.

Another aspect of the invention provides a polynucleotide comprising at least 20 bases of the human CPB2 gene and comprising an allelic variant selected from any one of the following:

| Position in | Variant |
|---|---|
| SEQ ID NO 1 | allele |
| 310 | T |
| 549 | G |
| 682 | A |
| 772 | T |

According to another aspect of the present invention there is provided a polynucleotide comprising at least 20 bases of the human CPB2 gene and comprising an allelic variant selected from any one of the following:

| Position in | Variant |
|---|---|
| SEQ ID NO 1 | allele |
| 310 | T |
| 524 | G |
| 549 | G |
| 682 | A |
| 697 | T |
| 772 | T |

Fragments are at least 17 bases, more preferably at least 20 bases, more preferably at least 30 bases.

A preferred polynucleotide comprises any one of the following six CPB2 haplotypes with reference to positions in SEQ ID NO 1:

| | 310 | 524 | 682 | 697 | 772 | 1059 |
|---|---|---|---|---|---|---|
| Haplotype | allele | allele | allele | allele | allele | allele |
| 1 | C | G | A | C | C | T |
| 2 | C | A | G | C | C | T |
| 3 | C | G | A | C | T | C |
| 4 | T | G | A | T | C | T |
| 5 | C | G | A | C | C | C |
| 6 | C | A | G | C | C | C |

The invention further provides a nucleotide primer which can detect a polymorphism of the invention.

According to another aspect of the present invention there is provided an allele specific primer capable of detecting a CPB2 gene polymorphism at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a CPB2 gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a CPB2 gene polymorphism at one of the positions defined herein.

According to another aspect of the present invention there is provided a kit comprising an allele specific oligonucleotide probe of the invention and/or an allele-specific primer of the invention.

The kit may comprise appropriate packaging and instructions for use in the methods of the invention. Such kit may further comprise appropriate buffer(s) and polymerase(s) such as thermostable polymerases, for example taq polymerase.

In another aspect of the invention, the polymorphisms of this invention may be used as genetic markers in linkage studies. This particularly applies to the polymorphisms of relatively high frequency. The CPB2 gene is on chromosome 13q14.11. Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWP:Ag levels: identification of three novel single nucleotide polymorphisms in the vWF gene promoter. Blood (1999) 93:4277-83, Keightley A M, Lam Y M, Brady J N, Cameron C L, Ullicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

Another aspect of the invention provides a method of treating a human in need of treatment with a CPB2 drug in which the method comprises:

i) detection of a polymorphism in CPB2 in the human, which detection comprises determining the sequence of the human at one or more of the following positions:
positions 310, 549, 682 and 772 in CPB2 polynucleotide as defined by the position in SEQ ID NO: 1; and
position 177 in CPB2 polypeptide defined by position in SEQ ID NO: 2
   and determining the status of the human by reference to polymorphism in CPB2; and

ii) administering an effective amount of the drug.

According to another aspect of the present invention there is provided a method of treating a human in need of treatment with a CPB2 drug in which the method comprises:

i) detection of a polymorphism in CPB2 in the human, which detection comprises determining the sequence of the human at one or more of the following positions:
positions 310, 524, 549, 682, 697 and 772 in CPB2 polynucleotide as defined by the position in SEQ ID NO: 1; and
positions 169 and 177 in CPB2 polypeptide defined by position in SEQ ID NO: 2.
   and determining the status of the human by reference to polymorphism in the CPB2 gene; and

ii) administering an effective amount of the drug.

Preferably determination of the status of the human is clinically useful. Examples of clinical usefulness include deciding which drug or drugs to administer and/or in deciding on the effective amount of the CPB2 drug or drugs. The reader is referred to the following PCT patent applications from AstraZeneca relating to compounds acting at CPB2: WO 00/66152, WO 00/66550 and WO 00/66557. The term “CPB2 drug” means that drug affecting CPB2 in humans is an important part of a drug exerting its pharmceutical effect in man.

According to another aspect of the present invention there is provided use of a CPB2 drug in preparation of a medicament for treating a disease in a human detected as having a polymorphism at one or more of the positions defined herein. Preferably the disease is cardiovascular.

According to another aspect of the present invention there is provided a pharmaceutical pack comprising CPB2 drug and instructions for administration of the drug to humans tested for a polymorphism at one or more of the positions defined herein.

Another aspect of the invention provides an allelic variant of human CPB2 polypeptide comprising a glycine at position 177 of SEQ ID NO 2; or a fragment thereof comprising at least 10 amino acids provided that the fragment comprises at least one allelic variant.

D at 177 is conserved between man and mouse and is part of a 26 amino acid sequence conserved between man and mouse suggesting that this is a functionally important site. Substitution of G for D substitutes a small, uncharged amino acid in place of a larger negatively charged amino acid in a conserved region which is expected to affect properties of the protein.

According to another aspect of the present invention there is provided an allelic variant of human CPB2 polypeptide comprising at least one of the following:
an alanine at position 169 of SEQ ID NO 2;
a glycine at position 177 of SEQ ID NO 2;
or a fragment thereof comprising at least 10 amino acids provided that the fragment comprises at least one allelic variant.

Fragments of polypeptide are at least 10 amino acids, more preferably at least 15 amino acids, more preferably at least 20 amino acids.

According to another aspect of the present invention there is provided an antibody specific for an allelic variant of human CPB2 polypeptide as described herein.

Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind the allelic variant of CPB2 with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394(1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a kit comprising an antibody of the invention.

According to another aspect of the invention there is provided a human CPB2 polypeptide comprising one of the following four combinations of allelic variant determined amino acids based on positions identified in SEQ ID NO: 2:

| | |
|---|---|
| 1 | thr169/thr347 |
| 2 | ala169/thr347 |
| 3 | thr169/ile347 |
| 4 | ala169/ile347 |

According to another aspect of the invention there is provided a polynucleotide which encodes any of the four combinations of allelic variant human CPB2 polypeptide as defined above.

References

Eaton, D. L.; Malloy, B. B.; Tsai, S. P.; Henzel, W.; Drayna, D.: Isolation, molecular cloning, and partial characterization of a novel carboxypeptidase B from human plasma. J. Biol. Chem. 266,21833-21838, 1991.

Tsai, S. P.; Drayna, D: The gene encoding human plasma carboxypeptidase B (CPB2) resides on chromosome 13. Genomics 14, 549-550, 1992.

Vanhoof, G.; Wauters, J.; Schatteman, K.; Hendriks, D.; Goossens, F.; Bossuyt, P.; Scharpe, S.: The gene for human carboxypeptidase U (CPB2)—a proposed novel regulator of plasmingogen activation—maps to 13q14.11. Genomics 38:454-455, 1996.

Bazjar, L.; Morser, J.; Nesheim, E.; TAFI, or plasma procarboxypeptidase B, couples the coagulation and fibrinolytic cascades through the thrombin—thrombomodulin complex. J. Biol. Chem. 271, 16603-16608, 1996.

The invention will now be illustrated but not limited by reference to the following Examples. All temperatures are in degrees Celsius.

In the Examples below, unless otherwise stated, the following methodology and materials have been applied.

AMPLITAQ™ available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or “Current Protocols in Molecular Biology”, Volumes 1-3, Edited by F M Asubel, R Brent & R E Kingston, published by John Wiley, 1998.

Electropherograms were obtained in a standard manner: data was collected by ABI377 data collection software and the wave form generated by ABI Prism sequencing analysis (2.1.2).

### EXAMPLE 1

Identification of Polymorphisms

1.1 Methods

The genomic structure of CPB2 was determined by BLAST analysis using the cDNA sequence against human genomic sequence. This indicated 11 exons. Primers were designed in the flanking intron sequence to amplify individual exons from genomic DNA of 29 unrelated Europeans. The PCR primers were tagged with either M13f or M13r sequence. Individual PCR products were sequenced by dye-terminator sequencing in both directions using M13f or M13r primers.

1.2 Results

| Position in | | | | Amino acid |
|---|---|---|---|---|
| SEQ ID | Reference | Variant | Variant allele | Change, position in |
| NO 1 | allele | allele | Frequency | SEQ ID NO 2 |
| 310 | C | T | 13/56 | Silent asp97 |
| 524 | A | G | 31/46 | Thr169ala |
| 549 | A | G | 1/46 | Asp177gly |
| 682 | G | A | 35/50 | Silent pro 221 |
| 697 | C | T | 11/50 | Silent asp226 |
| 772 | C | T | 4/56 | Silent ile 251 |

D at 177 is conserved between man and mouse and is part of a 26 amino acid sequence conserved between man and mouse suggesting that this is a functionally important site. Substitution of G for D substitutes a small, uncharged amino acid in place of a larger negatively charged amino acid, which is expected to affect properties of the protein.

This analysis also confirmed TSC SNP TSC0985620 as a genuine polymorphism, substituting T for C at position 1059 and resulting in amino acid change thr347ile. The frequency of the T allele in 40 Europeans was 78%.

### EXAMPLE 2

Haplotypes

The polymorphisms at 310, 524, 682, 697, 772, and 1059 are inherited as 6 common haplotypes.

| | 310 | 524 | 682 | 697 | 772 | 1059 |
|---|---|---|---|---|---|---|
| Haplotype | allele | allele | allele | allele | allele | allele |
| 1 | C | G | A | C | C | T |
| 2 | C | A | G | C | C | T |
| 3 | C | G | A | C | T | C |
| 4 | T | G | A | T | C | T |
| 5 | C | G | A | C | C | C |
| 6 | C | A | G | C | C | C |

These haplotypes indicate that there are 4 common forms of the CPB2 protein: Thr169/thr347 (50%), ala169/thr347 (21%), thr169/ile347 (19%) and ala169/ile347 (10%).

1. A method for the detection of a polymorphism in CPB2 in a human, which method comprises determining the sequence of the human at at least one of the following polymorphic positions:
positions 310, 549, 682 and 772 in CPB2 polynucleotide as defined by the position in SEQ ID NO: 1; and
position 177 in CPB2 polypeptide defined by position in SEQ ID NO: 2.

2. A method according to claim 1 which comprises detection of any one of the following six haplotypes:

| | 310 | 524 | 682 | 697 | 772 | 1059 |
|---|---|---|---|---|---|---|
| Haplotype | allele | allele | allele | allele | allele | allele |
| 1 | C | G | A | C | C | T |
| 2 | C | A | G | C | C | T |
| 3 | C | G | A | C | T | C |
| 4 | T | G | A | T | C | T |
| 5 | C | G | A | C | C | C |
| 6 | C | A | G | C | C | C |

3. Use of a method as defined in claim 1 to assess the pharmacogenetics of a CPB2 drug.

4. A polynucleotide comprising at least 20 bases of the human CPB2 gene and comprising an allelic variant selected from any one of the following:

| Position in | Variant |
|---|---|
| SEQ ID NO 1 | allele |
| 310 | T |
| 549 | G |
| 682 | A |
| 772 | T |

5. A polynucleotide according to claim 4 comprising any one of the following CPB2 haplotypes with reference to positions in SEQ ID NO 1:

| | 310 | 524 | 682 | 697 | 772 | 1059 |
|---|---|---|---|---|---|---|
| Haplotype | allele | allele | allele | allele | allele | allele |
| 1 | C | G | A | C | C | T |
| 2 | C | A | G | C | C | T |
| 3 | C | G | A | C | T | C |
| 4 | T | G | A | T | C | T |
| 5 | C | G | A | C | C | C |
| 6 | C | A | G | C | C | C |

6. A nucleotide primer which can detect a polymorphism as defined in claim 1.

7. An allele specific primer capable of detecting a CPB2 gene polymorphism defined in claim 1.

8. An allele-specific oligonucleotide proA polynucleotide comprising at least 20 bases of the human CPB2 gene and comprising an allelic variant selected from any one of the following:

| Position in | Variant |
|---|---|
| SEQ ID NO 1 | allele |
| 310 | T |
| 549 | G |
| 682 | A |
| 772 | T |

9. Use of a CPB2 polymorphism as defined in claim 1 as a genetic marker in a linkage study.

10. A method of treating a human in need of treatment with a CPB2 drug in which the method comprises:
i) detection of a polymorphism in CPB2 in the human, which detection comprises determining the sequence of the human at one or more of the following positions:
positions 310, 549, 682 and 772 in CPB2 polynucleotide as defined by the position in SEQ ID NO: 1; and
position 177 in CPB2 polypeptide defined by position in SEQ ID NO: 2
and determining the status of the human by reference to polymorphism in CPB2; and
ii) administering an effective amount of the drug.

11. An allelic variant of human CPB2 polypeptide comprising a glycine at position 177 of SEQ ID NO 2;
or a fragment thereof comprising at least 10 amino acids provided that the fragment comprises at least one allelic variant.

12. An antibody specific for an allelic variant of human CPB2 polypeptide as defined in claim 11.
